(19) United States
(12) Patent Application Publication
(10) Pub. No.: US 2008/0234642 A1
(43) Pub. Date: Sep. 25, 2008

(54) Male hygenic device

(76) Inventors: Frank V. Patterson, Exeter, NH (US); Denis LaBombard, Georgetown, MA (US); Kim A. Figuerido, Providence, RI (US)

Correspondence Address: FRANCIS H. KIRKPATRICK;KIRKPATENT, 37 CLOVER HILL DR., CHELMSFORD, MA, 01824-2611, US

(21) Appl. No.: 12/077,945

(22) Filed: Mar. 22, 2008

Related U.S. Application Data
(60) Provisional application No. 60/919,803, filed on Mar. 23, 2007.

(51) Int. Cl. A61M 1/00; A61F 5/44

(52) U.S. Cl. 604/319; 604/347; 604/349

(57) ABSTRACT

An improved device for male hygiene intended for collection of urine is described. An absorbent pad comprises a polymer that forms a gel upon contact with water, which is enclosed in a water-permeable, corrugated plastic pouch. The pouch is contained, optionally by adhesive, in a flexible stretchable water-impermeable sheath, which in use is placed over at least the glans of the penis. Because of the corrugations, the absorbent pad curls up into a cup shape when it swells upon absorption of urine, maintaining the device in a compact configuration for inconspicuous functioning. The absorbent pad may be supplied separately from the flexible sheath.

### PRIORITY

This invention claims the benefit of the priority of provisional application U.S. 60/919,803, filed Mar. 23, 2007, which is hereby incorporated by reference in its entirety where permitted.

### FIELD OF THE INVENTION

The present invention relates generally to devices for absorbing small uncontrolled releases of urine from males, and method for making and for using the same. The device comprises a small flexible pad, comprising a pouch of flexible, water-permeable material containing a non-soluble superabsorbent material for water. The pad is preferably planar and preferably corrugated, so that it can curl to conform to the shape of the penis when it absorbs urine. The pad is held in place by a condom-like, easily stretchable sheath, to which the pad may optionally be bonded. After use, or after wearing for a prolonged period, the pad is discarded, optionally while encased within the sheath. The device is easy to manufacture and can be affordable for users subject to small volume uncontrolled urine releases.

### BACKGROUND OF THE INVENTION

The invention relates to a device for male hygiene, comprising an absorbent pad that is conformal to the tip of the penis and a condom-like sheath which stabilizes the pad on the penis without the use of adhesive strips.

The use of absorbing devices for mildly incontinent males is known in the art. More specifically, such devices heretofore devised and utilized for the purpose of absorbing “dribbling”, or leakage of relatively small amounts of urine, consist of a variety of structural configurations. The following patents provide examples of such devices.

U.S. Pat. No. 2,873,740 to Wainwright discloses a penile shaped sheath with a belt, a cup and a liner. U.S. Pat. No. 4,673,401 to Jensen and Ferguson discloses a male incontinence device. U.S. Pat. No. 4,790,835 to Elias discloses a urinary male tubular type resilient device with absorbent material, with holes in the tip for ventilation. U.S. Pat. No. 4,863,448 to Berg discloses an absorbent cap-like device with adhesive. U.S. Pat. No. 4,886,509 to Mattsson describes a multilayered bag with absorbent, held on by tape. U.S. Pat. No. 4,944,733 to Casaie discloses a diaper for use in toilet training male children or for use by incontinent male adults. U.S. Pat. No. 4,675,012 to Rooyakkers discloses a method of forming an absorbent genitalia pouch for incontinent males. U.S. Pat. No. 5,009,649 to Goulter et al discloses an expandable banded male urinary incontinence condom and supporting undergarment.

U.S. Pat. No. 5,074,853 to Bryant discloses a male incontinence diaper. U.S. Pat. No. 5,276,085 to Conway and US 2006/0229576 to Conway et al describe a tubular sheath with absorbent in the sheath or in a detachable receptacle at the distal end of the sheath, adhered to the penis by adhesive when the top is unrolled, and made by a complex, 12 step manufacturing method. U.S. Pat. No. 5,643,235 to Figuerido discloses a tubular device with adhesive tabs and absorbent materials within the entire interior of the tubular device. U.S. Pat. No. 5,695,485 to Duperret et al discloses a pouch with 300 cc liquid capacity and multilayered absorbent, retained in underwear by adhesive strips. U.S. Pat. No. D399,952 to McMillian discloses a strapping system for an incontinence sheath. U.S. Pat. Nos. 6,129,718 and 6,338,279 to Wada disclose absorbent folded sheets with elastic members. U.S. Pat. No. 6,131,575 to Lenker et al. discloses an adhesively secured pad for the glans of the penis. U.S. Pat. No. 6,248,096 to Dwork and Elson and U.S. Pat. No. 6,113,582 to Dwork discloses an overlapping absorbent wrap with an adhesive and other retention means. U.S. Pat. No. 6,336,919 to Davis and Mitchell discloses an absorbable wrapable shield with fastener strips. U.S. Pat. No. 6,479,726 to Cole is similar. U.S. Pat. No. 6,419,665 to Cohen discloses a male undergarment with a tubular absorbent receptacle. U.S. Pat. No. 6,949,090 to Leers et al. discloses a tubular sheath with an elastic band and a distal bulbous area with celluosic absorbent. US 2001/0031933 to Canon has a permeable tube contacting the penis and a large bladder with a sponge-like absorbent. US 2006/02060707 A1 to Larsen discloses an integral sheath with absorbent area with adhesive tabs for securement. US 2003/0023222 to Chen is generally similar, but the tubular sheath is not permeable and is secured by hook/loop closures (e.g. Velcro®). U.S. Pat. No. 4,601,716 to Smith has a cap shaped bag with absorbent held below the glans by an elastic band or hook/loop closure. US. 2002/0188265 A1 to Law discloses a penile cap with cellulosic absorbent held in place by an elastic band.

While the above devices address particular objectives and requirements related to controlling leakage of urine in males, the aforementioned patents do not describe the device and method which are the objectives of the present invention. Moreover, despite the many devices cited above, spanning over 45 years of patenting activity and development for this type of device, there are no devices of the type described in this invention presently on the market, despite a deeply felt need. It appears that none of the devices patented to date have delivered the required performance, or have been too expensive.

Hence, the device for the penis according to the present invention substantially departs from the conventional concepts and designs of the prior art, and in doing so provides an apparatus primarily developed for the purpose, one that does not rely on adhesive for the stabilization of the device in place, or for leak resistance, or associated garment design to stabilize the device. It furthermore avoids the use of woven and non-woven materials (including cellulosics) for barrier functions. and absorbency, because these tend to be bulky as well as difficult to manufacture in a shape conducive for penile anatomical comfort.

Therefore, it can be appreciated that there exists a continuing need for a new and improved urine-absorptive device for the penis of a male suffering from a mild incontinence or leakage of urine. Such a device needs to be ergonomic, secure, absorbent, and comfortable over the penis both before and after absorption of urine.

### SUMMARY OF THE INVENTION

In view of the foregoing disadvantages inherent in the types of devices for incontinent males now known in the art, the present invention provides an improved device for absorbing small amounts of urine emitted from the penis of a male, whether due to incontinence or stress. As such, the general purpose of the present invention is to provide a new and improved urine-absorption device for the penis of a male, which is structurally suitable to perform the required functions, as well as methods for the manufacture and use of the improved device.

The improved device of the invention comprises, in one aspect, a tubular device for the penis of a male. The inventive device is a comfortable device having a form fitting absorbent pad that easily fits over and is stable on the penis for moisture absorption, and a tubular, stretchable sheath for approximation of the pad to the penis. The device of the invention is small enough to be to comfortably worn under a pair of briefs and trousers, both before and after serving its function. Moreover, the device is truly convenient to apply, and remains in the place without adhesives while offering leak protection. The device is easy to remove and replace, allowing for normal urination.

To attain this, the present invention in preferred embodiments consists essentially of an absorbent pad within a tube. The tube has a closed hemispherical distal end and an open generally circular proximal end. The flexible tube is thin walled (preferably less than about 0.005 inches in wall thickness), and has a diameter ranging from about 0.75 inch to 2 inches. The range is wider than with condoms because the fit is preferably slightly loose; it may be necessary to offer various diameters for maximal user comfort. The tube has a length that is selectable between about 1 to 7½ inches. The thin tube is easily rolled up, as with a condom, and can be unrolled to whatever extent is necessary to retain the device's pad in proximity to the urethra. The tube is sufficiently elastic to easily be distended, with low applied force, and can retain its position on a flaccid penis without restricting blood circulation therein.

The absorbent pad resides in the distal end of the tube during use. The pad is a pouch containing a super absorbent material, which may be partially or substantially affixed to the inner surface of the pouch. Both of the pouch surfaces (distal and proximal) are concentrically or spirally corrugated, so that pad can change in shape from planar to hemispherical or tubular when applied over the distal end of the penis, without requiring the application of significant force to deform the pad or to maintain it in the more tubular configuration. For the purposes of this invention, a super absorbent material is a material that can absorb at least 10 times its weight in water or urine, preferably more, while remaining in a solid, gelled or semi-gelled state, i.e., without dissolving in the aqueous solution. Many such materials are known. Typical super absorbent materials are based on polymers, typically crosslinked or otherwise rendered water-insoluble, containing a high density of hydrophilic groups, such as hydroxyl and charged groups.

The proximal surface of the pad is rendered at least partially porous or otherwise water-permeable, to allow liquid passage through the pad wall into the interior of the pad, where the liquid will combine with the superabsorbant polymer to be stabilized, preferably as a gel. Once the liquid has combined or hydrated with the superabsorbent polymer and formed a gel, the liquid is immobilized and can not return across the water-permeable layer to pose a potential for leakage from the tube or irritation to the skin. Optionally both surfaces may be permeable.

It is therefore an object of the present invention to provide the user with a device that offers security from inadvertent urine release without the use of straps, tubes, adhesives, remote collection bags or specialized clothing. It is another object of the present invention to provide a new and improved tubular device for absorbing urine emitted by an incontinent male, which device may be easily and efficiently manufactured and marketed. It is a further object of the present invention to provide a new and improved tubular device for the penis of an incontinent male which is of durable and reliable construction.

Another object of the present invention is to provide a new and improved tubular device for the penis of an incontinent male which is has a low cost of manufacture with regard to both materials and labor, and which accordingly is then amenable to low prices of sale to the consuming public, thereby making such tubular device for the penis of an incontinent male economically available to the buying public.

Another object of the present invention is to provide a tubular device for the penis of an incontinent male that is ergonomically positionable and removable over the penis.

Another object of the invention is to provide a device for absorbing urine emitted by a male, the device comprising an absorbent pad for use in absorbing urine discharged from the penis, wherein the absorbent pad comprises two corrugated films, at least one of which is liquid permeable, joined to form a closed pouch, and further comprising a superabsorbent polymer contained within the pouch; and an elastomeric tubular sheath that secures said absorbent pad in position over the glans of the penis. The absorbent pad may be corrugated so that the corrugated pad folds to conform to the glans of the penis. The liquid permeability of the pad may be due to perforations formed in at least one of the films, or due to inclusion at least one of water-soluble materials and water-swellable materials in at least one of the films. The films are preferably joined by one or more of sonic welding, heat welding, and adhesive bonding. The superabsorbent may be at least partially adhered to the inner surface of the pouch.

The elastomeric tubular sheath typically has a diameter in the range of about 0.75 to 2.0 inches, and may be made available in multiple different diameters, to accommodate varying sizes of penis and/or various states of turgor of the penis. The pad is adhered to the sheath in one embodiment; in another embodiment, the sheath and pad are not adhered to each other, and in one embodiment the sheath and the pad are separately packaged.

Another object of the invention is to provide a method for controlling small leaks of urine by a male for a period of time, the method comprising the steps of application of a device to the penis, wherein the device is characterized in comprising a resilient sheath and an absorbent pad, and the pad is characterized in comprising a water-permeable film which encloses a superabsorbent; leaving the device in place during the period when control of urine leaks is desired; and removing and disposing of the device at a time point selected from the end of the period, and the time of absorption of urine by the device. In one embodiment, the device may be further characterized in that the absorbent pad is adhered to the sheath. In another embodiment, the absorbent pad is not adhered to the sheath, and in a third embodiment, the absorbent pad and the sheath are separately packaged.

Another object of the invention is to provide a method of manufacturing a device for absorbing small quantities of urine from a male, wherein the method comprises the steps of providing an elastomeric sheath, the sheath being characterized in being readily stretchable so as to accommodate the penis in varied degrees of turgor; and providing a highly flexible pad comprising two films and a superabsorbent material enclosed between them, suitable for prolonged contact with the penis; and placing the pad within the sheath to form the complete device. The placement of the pad in the sheath may occur prior to the application of the elastomeric sheath to the penis, or the application of the elastomeric sheath to the penis may occur after the placement of the pad on the penis. The placement of the pad in the sheath may occur at a factory. The placement of the pad in the sheath may be made by the end user or a person assisting the end user.

In any of the above embodiments, the pad may be corrugated, thereby increasing its flexibility.

These together with other objects of the invention, along with the various features of novelty which characterize the invention, are pointed out with particularity in the claims annexed to and forming a part of this disclosure. For a better understanding of the invention, its operating advantages and the specific objectives attained by its uses, reference should be had to the accompanying drawings and descriptive matter in which there are illustrated preferred embodiments of the invention.

### BRIEF DESCRIPTION OF THE DRAWINGS

FIG. 1 shows a cross-section view of an absorbent pad (6) in planar form.

FIG. 2 shows a cross section of an absorbent pad (6) within a condom-like device (5) prior to application to the penis.

FIG. 3 shows a cross section of an absorbent pad (6) within a condom-like device (5) in the installed shape conforming to the glans of the penis. This illustrates how the concentric corrugations of increasing diameter of the absorbent pad (6) elongate during installation and allow the absorbent pad to conform to the shape of the end of the penis.

### DETAILED DESCRIPTION OF THE INVENTION

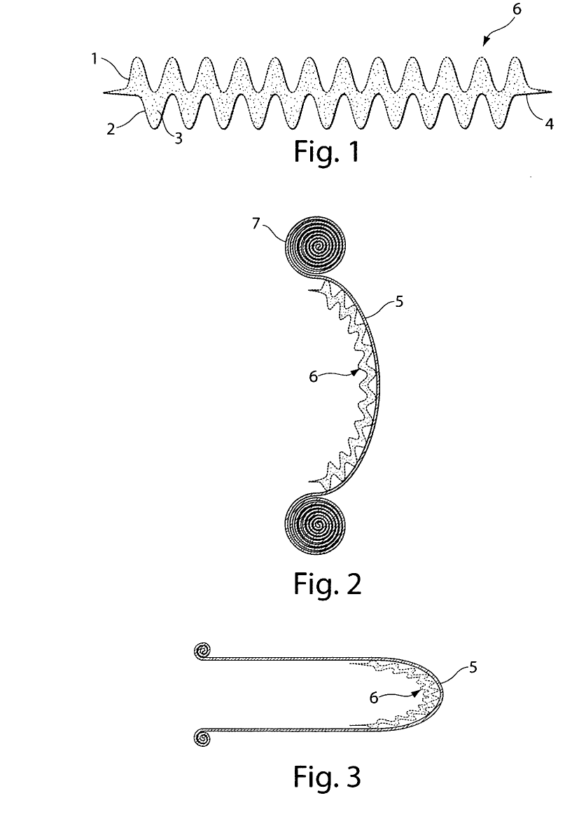

The hygienic device illustrated in FIGS. 1, 2 and 3 is a dribble protector for males experiencing uncontrolled or involuntary incontinent episodes wherein liquid discharges, typically of less than about 10 ml, require containment before clothing contact. Such low volume discharges are commonly encountered with patients undergoing medical transurethral procedures involving the bladder and prostate in which full control of bladder voiding is temporarily compromised and for which a simple method for collecting discharges is desired thereby eliminating the bulk and discomfort of diaper devices. In addition, sphincter control may be generally weaker in some men, especially elderly men, and various circumstances including stress may benefit from a simple means of absorbing small leakages.

FIG. 1 shows a cross-section view of an absorbent pad (6) in planar form. The absorbent pad 6 comprises two films (1 and 2), with concentric or spiral formed corrugations. The films are preferably of a low durometer rating (e.g. (25-80 Shore A), for example of polyurethane, so that they are flexible and comfortable both in the wet and dry states. In between the corrugations is a supply of superabsorbent material 3. The two films are sealed together on their perimeter to form a compartment which will retain the superabsorbent material within the structure of the pad even when it is swollen with liquid. The films may also be at least partially joined at the corrugations. One or both of the films 1 and 2 are water-permeable, as described in detail below.

FIG. 2 shows a cross section of an absorbent pad (6) within a condom-like tubular sheath (5) prior to application to the penis. Most of the material of the tubular sheath is rolled up (7), as with a condom. The tubular device is not water-permeable.

FIG. 3 shows a cross section of an absorbent pad with a tubular sheath 5 in the installed shape conforming to the glans of the penis (not illustrated). This illustrates how the concentric corrugations of the increasing diameter of the absorbent pad elongate and unfold during installation and allow the absorbent pad to conform to the shape of the end of the penis.

The tubular sheath 5 is similar in construction, materials and feel to a commercially available condom. For example, it may be made of rubber latex similar to a standard condom. Because any emitted urine is promptly immobilized by the pad, the tubular sheath does not need to meet the form fitting and security requirements of condoms on an engorged penis, but merely needs to hold the absorbent pad in place on a flaccid penis with the added support of under briefs. Hence, it may be thinner and/or more easily stretched than a standard condom, which may be preferred for comfort. In some embodiments, resilient polyurethanes can be used in the construction of the sheath, and other resilient and flexible synthetic or natural materials may be suitable.

Suitable materials for the construction of the film forming the wall of the absorbent pad are polymers that are common for construction of soft, film based, skin contact devices. There are numerous polymers fitting the description. One example, which we have used in prototyping, is thermoplastic polyurethane (DOW “Pellathane” and Noveon Estane). Such materials are easily converted into thicknesses of less than 0.005 inches and then formed with corrugations using traditional extrusion and thermoforming manufacturing methods to form the distal and proximal elements of the absorbent pad. After filling with superabsorbent (see below), the thermoformed elements are joined on their perimeters, and optionally at least partially in the troughs of the corrugations, to form a closed compartment. Joining can be done with any of numerous traditional commercially available heat sealing or welding methods. Alternatively, the joining can be done with adhesives. It is preferable to form the corrugations before filling the pads with superabsorbents and then joining the films at the edges. However, in high volume production, the operations can be done concurrently in one production line for efficiency.

The surfaces of the film, including the inside surfaces of the perforations, should be manufactured or treated so that they are wettable (hydrophilic), to allow the liquid urine to form a low contact angle with the surface, thereby favoring its passage to and across the perforations (described below), and into the interior of the pad. Once the moisture is inside, the super absorbent material will wick the liquid into contact with the absorbent, and preferably will form an immobilized gel structure. One aspect of the invention is efficient urine transport from the skin surface into the pad, in order to achieve a state of comfort and dryness on the skin surface.

Perforations through the film allow liquid to pass from the urethra into the inner space of the absorbent pad, where the superabsorbent (3) forms a hydrated complexes with the liquid and traps the liquid, preferably as a gel. The perforations in the film may be in one or both films and preferably at least on the proximal side in contact with the penis. Perforations on the distal side of the absorbent pad serve to allow urine entry into the pad that has bypassed the proximal surface. It is preferable that the perforations allow a fast route for discharged urine to travel to the superabsorbent. The speed of that transport process is favored by a high density of perforations, preferably with diameters of less than about 0.020 inch (0.5 mm). The perforations are created by any conventional means, including mechanical piercing means and laser piercing.

Alternatively, or in addition, porosity can be provided by incorporation of an inclusion (e.g., salt, or water-swellable polymer) within the polymer film, so that on exposure to water, the inclusion is dissolved away, or hydrates and forms a passage for water through the film. In another alternative, the perforate film may be provided in the form of a woven or nonwoven fabric of a thermoplastic material, rendered sufficiently hydrophilic to conduct water therethrough by any commercial procedure or combination thereof, including without limitation coating, etching and heating. The fabric should be very pliable and flexible, for comfort, but also have fibers that are hydrophilic enough on their surfaces to allow water conduction, but which preferably do not absorb water into the fibers. For example, rapid drying polyethylene sports fabrics have such preferred properties.

In addition, one or more macroscopic holes through the pad, bounded by the material of the film, may be provided to facilitate access of the urine to the outside surface of the pad, to maximize absorption speed. For example, a hole could be placed in the center of the pad for this purpose (not illustrated). A diameter of 2-4 mm would be adequate for the purpose.

The superabsorbent (3) is preferably reasonably evenly dispersed within the pad and in close proximity to the perforations. One way to achieve this distribution is to embed the particles of superabsorbent 3 into the films 1 and/or 2. For example, the particles can be placed on or blown into the film when the film is in a molten or partially molten state during thermoforming. The molten film entraps the particles and renders them stationary and in close proximity to the perforations.

Suitable superabsorbent materials do not dissolve in water; rather, the material will chemically absorb and retain water-based fluids. The desired particle size, when the superabsorbent is particulate, ranges between approximately 50-850 microns. The particle size of the superabsorbent, and the size of the perforations (if they are open, vs. latent as with salt) need to be correlated so the powder will not leak out, unless the superabsorbent is bound to the film. Alternatively, a thin coherent layer of superabsorbent, manufactured by pressing, cross-linking, or other conventional procedure, can be placed between the structural films 1 and 2 during the manufacturing process. A low density of sealing through the absorbent layer would aid retention of the corrugated shape, when advantageous.

The superabsorbent can be any of a large variety of such materials, which are well known. As an example, a composite hydrogel, based on xanthan and poly(vinyl alcohol) and cross linked with epichlorohydrin, has been tested. Generally, the superabsorbents are long polymer chains, for example potassium polyacrylate, hydroxyl-derivatized cellulose, or other hydrophilic water-compatible polymer, which are linked together into a three-dimensional structure using crosslinking agents or structural equivalents thereto. Depending on the system, the crosslinking may be covalent, ionic, or based on amphiphilic polymers having both hydrophobic and hydrophilic regions.

During use the polymers will become swollen with urine. Hence, the polymers should contain no free components that would damage skin during prolonged contact. Materials qualified as USP or otherwise rated for prolonged skin contact are preferred.

### Operation of the Device

When urine is emitted within the device, the surface wicking action of the absorbent pad will channel the liquid away from the skin surface and into and through the pad's porous openings, and allow the liquid to be absorbed within the inner matrix of superabsorbant material. A preferred superabsorbent material takes up an aqueous based electrolyte (such as urine) by absorbing up at least about 10 times its weight in liquid, and preferably more. Superabsorbents absorbing 30 times their weight in liquid are known, for example crosslinked sodium acrylate. With such a material, a volume of only 0.35 cc of superabsorbent can contain and trap up to 10 cc of liquid during its hydration process. The superabsorbent transforms into a gel or gel-like structure during the hydration process, rendering the liquid bound and preventing any bulk mobility or migration back across the permeable porous boundary. The superabsorbent prevents the liquid from becoming mobile within the tubular device and escaping from the condom onto clothing.

The device is easily disposed of when capacity is reached. Before use, it can be conveniently carried by the user for future use due to its compact storage size, which may be less than about ¼ inch (6 mm) in thickness and less than about 2½ inches (70 mm) in diameter. The device is preferably packaged in disposable paper, foil or plastic film packaging, preferably with a tear open feature for easy access. When superabsorbent capacity is as much as 10 g/g, a pad capable of absorbing over 10 cc of fluid, together with a sheath, can weigh less than 3 grams.

In one version of the device, the pad and the sheath are connected, so that the pad is retained within the sheath during storage, use and disposal. Such a connectivity can be most easily obtained by placing the pad in the sheath and joining them by the use of heat or pressure, optionally accompanied by the use of an adhesive attached to one of the parts. Attaching a dot of adhesive to the center of the pad, for instance, would be straightforward. A contact adhesive would be suitable, even without heat or significant pressure; and preferably a contact adhesive would be selected so that the adhesiveness would not quickly dissipate in the presence of water or urine.

In another version, the pad and the sheath are not adhesively joined. The pad may be retained in the sheath by the act of rolling the sheath for compactness. For example, the distal (closed) end of the sheath could be at least the same diameter as, and preferably larger in diameter than, the proximal end of the sheath. Then a pad could be folded sufficiently to be inserted into the closed distal end of the sheath, and then the sheath can be rolled up, entrapping the pad in the distal portion of the device.

In a third version, the sheaths and pads can be provided together in a package, but not assembled together, and the customer can insert the pad into the sheath. Alternatively, the user can place the pad at the opening of the urethra on the glans of the penis, and unroll the sheath to cover and contain both the pad and the penis.

In a fourth version, the pads and the sheaths can be sold separately. In this version, the customer may choose to reuse sheaths, or to use conventional condoms for the same purpose, albeit perhaps at a lower level of comfort. These latter versions, if acceptable to the customer, reduce manufacturing and especially packaging costs, which may make the product more acceptable in terms of cost to potential customers on fixed incomes.

The important features of the invention have been described so that that the invention that may be understood and in order that the present contribution to the art may be appreciated. It is to be understood that the invention is not limited in its application to the details of construction and to the arrangements of the components set forth in the description or illustrated in the drawings. The invention is capable of being made in other embodiments and of being practiced and carried out in various ways. Hence, those skilled in the relevant art will appreciate that the conception upon which this disclosure is based may readily be utilized as a basis for the designing of other structures, methods and systems for carrying out the purposes of the present invention. It is important, therefore, that the claims be regarded as including such equivalent constructions insofar as they do not depart from the scope of the present invention. Also, it is to be understood that the phraseology and terminology employed herein are for the purpose of descriptions and should not be regarded as limiting. Only the claims are to be regarded as limiting the scope of the invention.

1. A device for absorbing urine emitted by a male, the device comprising:
an absorbent pad for use in absorbing urine discharged from the penis, wherein the absorbent pad comprises two corrugated films, at least one of which is water permeable, joined to form a closed pouch, and further comprising a superabsorbent polymer contained within the pouch; and
an elastomeric tubular sheath with an open proximal end and a closed distal end, that secures said absorbent pad in position over the glans of the penis.

2. The device of claim 1 wherein the absorbent pad is corrugated so that the corrugated pad folds to conform to the glans of the penis.

3. The device of claim 1 wherein the liquid permeability of the pad is due to perforations formed in at least one of the films.

4. The device of claim 1 wherein the liquid permeability of the pad is due to inclusion at least one of water-soluble materials and water-swellable materials in at least one of the films.

5. The device of claim 1 wherein the films are joined by one or more of sonic welding, heat welding, and adhesive bonding.

6. The device of claim 1 wherein the superabsorbent is at least partially adhered to the inner surface of the pouch.

7. The device of claim 1 wherein the sheath has a diameter in the range of about 0.75 to 2.0 inches.

8. The device of claim 1 wherein the pad is adhered to the distal end of the sheath.

9. The device of claim 1 wherein the sheath and pad are not adhered to each other.

10. The device of claim 1 wherein the sheath and the pad are separately packaged.

11. A method for controlling small leaks of urine by a male for a period of time, the method comprising the steps of application of a device to the penis,
wherein the device is characterized in comprising a resilient sheath with an open proximal end and a closed distal end, and an absorbent pad, and the pad is characterized in comprising a water-permeable film pouch which encloses a superabsorbent;
leaving the device in place during the period of time when control of urine leaks is desired;
and removing and disposing of the device at a time point selected from:
the end of the period; and
the absorption of urine by the device.

12. The method of claim 11 wherein the device is further characterized in that the absorbent pad is corrugated.

13. The method of claim 11 wherein the device is further characterized in that the absorbent pad is not adhered to the sheath.

14. The method of claim 11 wherein the application step includes the insertion, at the time off use, of the absorbent pad into the sheath before the application of the sheath to the penis.

15. A method of manufacturing a device for absorbing small quantities of urine from a male, the method comprising the steps of:
providing an elastomeric sheath, with an open proximal end and a closed distal end, wherein the sheath is characterized in being readily stretchable so as to accommodate the penis in varied degrees of turgor;
providing a highly flexible pad comprising two joined films and a superabsorbent material enclosed between them, suitable for prolonged contact with the penis; and
placing the pad within the sheath to form the complete device.

16. The method of claim 15 wherein the placement of the pad in the sheath occurs prior to the application of the elastomeric sheath to the penis.

17. The method of claim 15 wherein the application of the elastomeric sheath to the penis occurs after the placement of the pad on the penis.

18. The method of claim 16 wherein the placement of the pad in the sheath occurs at a factory.

19. The method of claim 16 wherein the placement of the pad in the sheath is made by the end user or a person assisting the end user.

20. The method of claim 15 wherein the pad is corrugated, thereby increasing its flexibility.
